(19) United States
(12) Patent Application Publication
(10) Pub. No.: US 2020/0038653 A1
(43) Pub. Date: Feb. 6, 2020

(54) MULTIMODAL CLOSED-LOOP BRAIN-COMPUTER INTERFACE AND PERIPHERAL STIMULATION FOR NEURO-REHABILITATION

(71) Applicants: UNIVERSITY OF FLORIDA RESEARCH FOUNDATION, INC., Gainesville, FL (US); THE UNITED STATES OF AMERICA, DEPARTMENT OF VETERANS AFFAIRS, Washington, DC (US)

(72) Inventors: Ranganatha SITARAM, Gainesville, FL (US); Janis Jaelynn DALY, Gainesville, FL (US); Mohit RANA, Gainesville, FL (US); Aniruddh RAVINDRAN, Gainesville, FL (US)

(21) Appl. No.: 16/065,581

(22) PCT Filed: Dec. 20, 2016
(86) PCT No.: PCT/US2016/067805

§ 371 Date: Jun. 22, 2018

Related U.S. Application Data
(60) Provisional application No. 62/270,852, filed on Dec. 22, 2015.

(51) Int. Cl. A61N 1/36; A61B 5/055; A61B 5/11; G06F 3/01

(52) U.S. Cl. CPC A61N 1/36003; A61B 5/055; G06F 3/015; A61N 1/36031; A61B 5/1123

(57) ABSTRACT

Brain impairment, for example due to stroke, is corrected in order to improve body movement. An fNIRS device is positioned over the motor cortex of non-impaired individuals, and blood oxygen in locations of the brain is analyzed to determine brain activity corresponding to a particular body movement. The movements are statistically analyzed, and are compared with fNIRS data gathered from a movement impaired individual attempting the same movement. A weighted value corresponding to the desired brain activity is generated using the statistical analysis, and is graphically displayed to the movement impaired individual during the attempts. This produces a feedback loop relating to the movement which can be repeated to produce brain plasticity in the impaired individual to facilitate the movement. Additionally, correct brain activity can be used to cause the application of an electrical signal to muscles of the body to produce the desired movement.

### CROSS-REFERENCE TO RELATED APPLICATION

This application claims the benefit of U.S. Patent Application No. 62/270,852, filed Dec. 22, 2015, the contents of which are incorporated herein by reference in their entirety.

### STATEMENT OF GOVERNMENT INTEREST

This invention was made with government support under Grant Nos. B9252-C, B9024S, and N2192P awarded by the U.S. Department of Veterans Affairs. The government has certain rights in the invention.

### FIELD OF THE DISCLOSURE

The disclosure relates to a system and method for promoting movement of the human body after brain impairment, and in particular, providing feedback incorporating brain imaging using fNIRS, as targeted using rtfMRI.

### BACKGROUND OF THE DISCLOSURE

Stroke is the leading cause of long-term disability worldwide and the number of affected people increases every year (WHO, 2011). Though promising work has shown some recovery of upper limb function, not all patients exhibit improvement (Lo et al 2011; Wolf et al 2009), and regrettably, there is no established method to restore upper limb function to normal following stroke.

Brain-computer Interfaces (BCIs) can, in real-time, record and decode some measurable brain neurophysiological signal and translate brain signal features into a format that may prove useful as a neural feedback system for motor learning in stroke survivors (Wolpaw 2012). Previous BCI studies, with non-invasive signal recording approaches, have used electroencephalography (Varkuti, Sitaram et al 2013; Daly et al 2009; Ang et al 2009) or magnetoencephaolography (MEG; Sachchet, Sitaram et al., 2012; Silvoni et al 2011; Daly and Sitaram, 2011 for reviews), and hemodynamic signals based on real time functional magnetic resonance imaging (rtfMRI) and functional near-infrared spectroscopy (fNIRS; Sitaram et al 2011; Daly and Sitaram 2011, for reviews).

Stroke survivors can gain control of brain activation associated with movement preparation and execution of motor tasks, using an EEG-based BCI (Varkuti, Sitaram et al 2013; Daly 2008; Daly et al 2009). Motor learning and instrumental conditioning of the brain responses using a closed-loop brain control interface have been proposed for aiding in restoring lost movement ability (Silvoni et al., 2011).

### SUMMARY OF THE DISCLOSURE

In an embodiment of the disclosure, a method of correcting brain impairment to improve body movement comprises analyzing at least one non-impaired brain of a subject during body movement using an rtfMRI device to target brain areas associated with those body movements; monitoring an impaired brain of a patient at the targeted brain areas using an fNIRS device and generating an output signal from the fNIRS device corresponding to brain activity in the targeted brain areas; and processing the output signal to produce visual feedback to the patient corresponding to positive or negative feedback relating to an extent of brain activity in the targeted brain areas.

In variations thereof, the method further includes stimulating muscles associated with the body movement using an electrical muscle stimulation device; the device is an FES stimulation device; the fNIRS device includes a signal generator, a photo multiplier, an amplifier, and an ADC; processing the output signal includes compensating for artifacts, including head movement, probe movement, and physiological noise; and/or processing the output signal includes converting HbO and HbR values provided by the FNIRS device into brain activity corresponding to brain activity within the targeted brain areas.

In further variations thereof, processing the output signal includes producing a graphic, moving visual indicator that is visible to the patient and which corresponds to positive and negative progress towards carrying out a desired brain activity corresponding to the body movement to be improved; the visual indicator resembles a thermometer; the method further includes processing the output signal to cause a signal from an FES device to stimulate muscles corresponding to brain activity in the targeted brain areas; and/or the FES device stimulates muscles to produce muscle movement and afferent nerve impulses which further stimulate the brain, thereby forming a closed-loop feedback system including the brain, the fNIRS, the FES device, and afferent nerves.

In another embodiment of the disclosure, a method for causing a desired body movement in a movement impaired individual comprises, with one or more non-movement impaired individuals: gather non-impaired feature data during a predetermined body movement of the one or more non-movement impaired individuals using multiple channel fNIRS to identify discriminative features of the fNIRS data corresponding to the predetermined body movement as changes in blood oxygen concentration in locations of the brain; select non-impaired data from the multiple channels, using the gathered non-impaired feature data, that optimally discriminate data corresponding to the predetermined body movements by considering relative entropy of the data; and input the selected non-impaired data into an SVM classification; and with the movement impaired individual: gather impaired feature data during attempts of the predetermined body movement by the movement impaired individual, using multiple channel fNIRS; select impaired data from the multiple channels by comparing gathered impaired feature data with the selected non-impaired data; apply the SVM classification to the selected impaired data to define weight values over time corresponding to a real time correlation of brain activity of the impaired individual with brain activity of the non-impaired individuals during the predetermined body movement; visually display the defined weight values as real time feedback for the impaired individual to be used by the movement impaired individual to change brain activity of the movement impaired individual to cause the predetermined body movement.

In a variation thereof, the method further includes, with one or more non-movement impaired individuals: gather non-impaired imagined feature data during imagination of the predetermined body movement not accompanied by the predetermined body movement, of the one or more non-movement impaired individuals using multiple channel fNIRS to identify discriminative features of the fNIRS data corresponding to the thoughts of the predetermined body movement as changes in blood oxygen concentration in areas of the brain; select non-impaired imagined data from the multiple channels, using the gathered non-impaired imagined feature data, that optimally discriminate data corresponding to the predetermined body movements by considering relative entropy of the data; and input the selected non-impaired imagined data into the SVM classification.

In other variations thereof, the method further includes using linear SVM classification to distinguish brain activity corresponding to the predetermined body movement carried out on the left side of the body and the right side of the body; the discriminative features of the fNIRS data is carried out using the formula:

$${f}^{n} ( k ) = {\sum }_{i ∈ I to {f}_{s}} Δ {HbO}_{i}^{n} ( k ) ; \qquad$$

selecting non-impaired data is carried out using the formula:

$$H ( ω \vert F ) = - {\sum }_{i = \{ 1 , 2 \}} p ( {ω}_{i} \vert F ) {log}_{2} p ( {ω}_{i} \vert F ) ;$$

and/or SVM classification of non-impaired data uses successive data points and the formula:

$${\{ N ; W \}}^{( r )} = \{ \begin{matrix}C ( {S}^{( r - 1 )} ) , & r = 2 \\ C ( \vert {S}^{( r - 1 )} ; {S}^{( r - 2 )} \vert ) , & r > 2\end{matrix} \} .$$

In still further variations thereof, a bias correction is applied to the gathered non-impaired feature data prior to applying the SVM classification; the method further includes stimulating muscles associated with the predetermined body movement using an electrical muscle stimulation device, when the weighted values correspond to the predetermined body movement; and/or the method is repeated over time to influence brain plasticity of the movement impaired individual with respect to the predetermined body movement.

In a further embodiment of the disclosure, a method for causing a desired body movement in a movement impaired individual comprises, with one or more non-movement impaired individuals: gather non-impaired feature data during a predetermined body movement of the one or more non-movement impaired individuals using multiple channel fNIRS positioned over the motor cortex; identify discriminative features of the fNIRS data corresponding to the predetermined body movement by evaluating time averages of changes in HbO concentration amount the multiple channels; select non-impaired data from the multiple channels, using the gathered non-impaired feature data, that optimally discriminate data corresponding to the predetermined body movements by considering relative entropy of the data; and input the selected non-impaired data into an SVM classification; and with the movement impaired individual: gather impaired feature data during attempts of the predetermined body movement by the movement impaired individual, using multiple channel fNIRS; select impaired data from the multiple channels by comparing gathered impaired feature data with the selected non-impaired data; apply the SVM classification to the selected impaired data to define weight values over time corresponding to a real time correlation of brain activity of the impaired individual with brain activity of the non-impaired individuals during the predetermined body movement; visually display the defined weight values as real time feedback for the impaired individual to be used by the movement impaired individual to change brain activity of the movement impaired individual to cause the predetermined body movement; and stimulating muscles associated with the predetermined body movement using an electrical muscle stimulation device, when the weighted values correspond to the predetermined body movement.

In a further embodiment of the disclosure, a system for correcting brain impairment to improve body movement, comprises: an rtfMRI device for analyzing at least one non-impaired brain of a subject during body movement to target brain areas associated with those body movements; an fNIRS device for monitoring an impaired brain of a patient at the targeted brain areas and generating an output signal from the fNIRS device corresponding to brain activity in the targeted brain areas; and processing means for processing the output signal to produce visual feedback to the patient corresponding to positive or negative feedback relating to an extent of brain activity in the targeted brain areas.

In variations thereof, the system further includes an electrical muscle stimulation device for stimulating muscles associated with the body movement; the device is an FES stimulation device; the fNIRS device includes a signal generator, a photo multiplier, an amplifier, and an ADC; processing the output signal includes compensating for artifacts, including head movement, probe movement, and physiological noise; and/or processing the output signal includes converting HbO and HbR values provided by the FNIRS device into brain activity corresponding to brain activity within the targeted brain areas.

In further variations thereof, processing the output signal includes producing a graphic, moving visual indicator that is visible to the patient and which corresponds to positive and negative progress towards carrying out a desired brain activity corresponding to the body movement to be improved; the visual indicator resembles a thermometer; the system further includes processing means for processing the output signal to cause a signal from an FES device to stimulate muscles corresponding to brain activity in the targeted brain areas; and/or the FES device stimulates muscles to produce muscle movement and afferent nerve impulses which further stimulate the brain, thereby forming a closed-loop feedback system including the brain, the fNIRS, the FES device, and afferent nerves.

In another embodiment of the disclosure, a system for causing a desired body movement in a movement impaired individual, comprises a multichannel fNIRS device having head mountable optodes including two groups of emitters and detectors, one group positionable over a motor cortex of the left hemisphere of a subject, and the other group positionable over a motor cortex of the right hemisphere of a subject; a computing device connected to the fNIRS device and a display device, the computing device including a processor configured for: gathering non-impaired feature data during a predetermined body movement of the one or more non-movement impaired individuals using the multiple channel fNIRS to identify discriminative features of the fNIRS data corresponding to the predetermined body movement as changes in blood oxygen concentration in locations of the brain; selecting non-impaired data from the multiple channels, using the gathered non-impaired feature data, that optimally discriminate data corresponding to the predetermined body movements by considering relative entropy of the data; and inputting the selected non-impaired data into an SVM classification; and with the movement impaired individual: gathering impaired feature data during attempts of the predetermined body movement by the movement impaired individual, using multiple channel fNIRS; selecting impaired data from the multiple channels by comparing gathered impaired feature data with the selected non-impaired data; applying the SVM classification to the selected impaired data to define weight values over time corresponding to a real time correlation of brain activity of the impaired individual with brain activity of the non-impaired individuals during the predetermined body movement; visually displaying the defined weight values upon the display device as real time feedback for the impaired individual to be used by the movement impaired individual to change brain activity of the movement impaired individual to cause the predetermined body movement.

In a variation thereof, the optodes in each group are arranged as alternating emitters and detectors in a checkerboard pattern.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 depicts a system of the invention including a device for acquiring a signal representative of brain activity corresponding to a desired body movement, processing of that signal, interactive training and presentation to the patient, and electrical stimulation of the patients muscles corresponding to the desired movement, in a feedback loop;

FIG. 2 depicts components of an embodiment of the system of FIG. 1, further including an rtfMRI component used to target brain activity associated with a particular movement, and a computer used to analyze signals, and provide feedback to the patient;

FIG. 3 depicts display screens presented to a patient in accordance with the disclosure, as well as a sequence and timing of training activities;

FIG. 4 depicts an individual wearing an fNIRS device in accordance with the disclosure;

FIG. 5 depicts an arrangement of light transmitters and receivers, in accordance with the disclosure, within the fNIRS device of FIG. 4;

FIG. 6 depicts a sequence of testing and training events in accordance with the disclosure;

FIG. 7 depicts a flow chart of a biofeedback process of the disclosure incorporating visual feedback elements;

FIG. 8 depicts example fNIRS channel outputs over time, during a series of the course of events depicted in FIG. 6;

FIG. 9A depicts the percentage classification accuracies for online binary (right v/s left motor tasks) classification for 7 subjects for Experiment 1;

FIG. 9B depicts the percentage classification accuracies for online binary (right v/s left motor tasks) classification for 7 subjects for Experiment 2;

FIG. 9C depicts a comparison of mean classification accuracies of motor imagery and execution when the classifier was trained on either motor imagery or motor execution; and

FIG. 10 depicts a computer system, some or all of the components of which can be used in carrying out the disclosure.

### DETAILED DESCRIPTION OF THE DISCLOSURE

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a basis for the claims and as a representative basis for teaching one skilled in the art to variously employ the present subject matter in virtually any appropriately detailed structure and function. Further, the terms and phrases used herein are not intended to be limiting, but rather, to provide an understandable description of the concepts.

The terms “a” or “an”, as used herein, are defined as one or more than one. The term plurality, as used herein, is defined as two or more than two. The term another, as used herein, is defined as at least a second or more. The terms “including” and “having,” as used herein, are defined as comprising (i.e., open language). The term “coupled,” as used herein, is defined as “connected,” although not necessarily directly, and not necessarily mechanically.

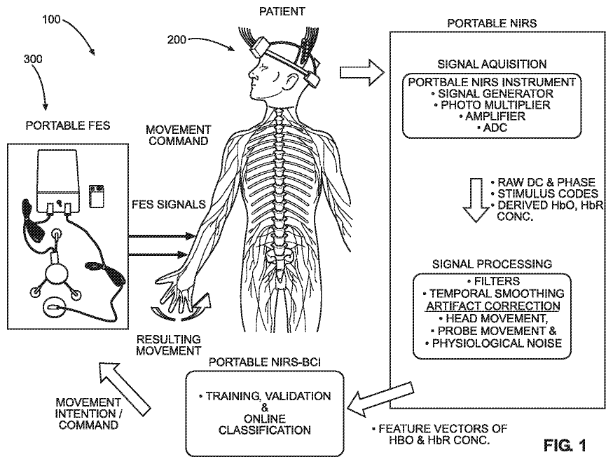

With reference to FIG. 1, the disclosure provides an optical brain-computer interface, and an orthosis for neurorehabilitation. System 100 of the disclosure provides a Brain Computer Interface (BCI) which includes a functional Near Infrared Spectroscopy (fNIRS) device, or fNIRS 200 for brain imaging, and a Functional Electrical Stimulation (FES) device, or FES 300, which provides feedback and electrical stimulation to the patient, as well as software which receives input from fNIRS 200, processes the input, provides output for visualization by the patient as described herein, and sends signals to FES 300, to produce a feedback loop of the disclosure.

The disclosure demonstrates that fNIRS 200 can be used as a brain imaging method with benefits including ease of setup and lack of interference from movement artifacts due to associated hardware, as compared, for example, to electroencephalography (EEG). fNIRS 200 also provides greater speed, portability and affordability as compared to functional Magnetic Resonance Imaging (fMRI). Due to portability and other advantages described herein, system 100 is useful for design and use with assistive devices, prosthetics, and robotic devices for applications in the field of rehabilitation. System 100 can further serve as an assessment and treatment tool in stroke patients with movement disorders. In an embodiment, the disclosure provides a system 100 which forms a closed-loop BCI with the fNIRS decoding patterns of hemodynamic brain signals which pertain to limb movements, combined with FES for afferent sensory feedback.

In another embodiment of the disclosure, system 100A provides a framework incorporating real-time functional Magnetic Resonance Imaging (rtfMRI) 400, together with functional Near Infrared Spectroscopy (fNIRS) 200 neurofeedback, with or without a peripheral control device such as robots, a Transcranial Magnetic Stimulation (TMS) system, Transcranial Direct Current Stimulation (TDCS), or Functional Electrical Stimulation (FES), hereinafter individually or collectively referred to as FES 300, for convenience. The above framework is applicable in two domains: scientific and clinical. System 100A is used as an exploratory tool for a hypothesis in the scientific domain, as well as a therapeutic intervention in the clinical setting. The findings are then consolidated into a rehabilitation protocol using system 100 with neurofeedback in the form of visual feedback, with or without peripheral stimulation, after which results are inferred for scientific purposes, whereas clinical tests are performed for clinical evidence. For treatment of a patient, a peripheral control device can be used, such as FES 300.

Functional near infrared spectroscopy (fNIRS) as used in accordance with the disclosure enables a low-cost, non-invasive manner for safely measuring brain activity using near infrared light. Further in accordance with the disclosure, patterns of fNIRS signals during motor execution and imagery are decoded and interpreted, thus making them useful to provide neural feedback for the purpose of motor learning. Minimal movement artifacts help in precise acquisition of fNIRS signals during motor tasks. Portable versions of system 100 can be used for a variety of neuro-rehabilitation protocols. Real-time functional magnetic resonance imaging (rtfMRI) 400 has shown capability in identifying patterns of brain activations for cognitive, emotional and motor tasks. Though precise in identifying patterns of brain activity, rtfMRI 400 is not practical in terms of cost and feasibility for motor rehabilitation protocols. Positioning of the patient and movement artifacts make it difficult to design neuro-rehabilitation protocols with rtfMRI BCIs. In accordance with the disclosure, information from rtfMRI 400 is used to coordinate and inform the use of fNIRS 300 and FES 300.

More particularly, in accordance with the disclosure, a two-phase procedure uses real-time functional magnetic resonance imaging (rtfMRI) 400 and functional near infrared spectroscopy (fNIRS) 200 sequentially in a neuro-rehabilitation protocol. Thus, the unique advantages of each method is combined. rtfMRI 400 is used as a closed loop system for extracting information from BOLD signals from regions of interest in real-time, so that this information can be provided to patients as contingent feedback to enable the control of brain activity and the capture of information. Due to a lower spatial resolution of fNIRS as compared with rtfMRI, the information regarding the brain areas involved in a particular task, obtained from rtfMRI, are used for targeting an exact brain area of interest when using fNIRS 200, thus making the use of fNIRS 200 more effective. Accordingly, rtfMRI 400 is used as a calibrating tool for fNIRS 200.

A co-registration algorithm, as understood within the art, is used to map the position of fNIRS optodes onto the structural image of the subject acquired at the scanner. Additionally, Functional Electrical Stimulation (FES), robotic prostheses and other assistive devices have been adopted for use in motor learning methods. Use of such devices is difficult in the rtfMRI environment. Accordingly, the invention exploits the portability and convenience of using fNIRS 200 in a convenient environment, to integrate brain activity with FES-assisted muscle activation to support performance of impaired movements, thus making it easier to implement motor rehabilitation protocols through the use of an fNIRS BCI of the disclosure.

The disclosure provides a patient-specific neuro-rehabilitation protocol that enables a customized therapy for each patient according to his or her learning curve. Since lesion induced plasticity, for example, varies in each patient, identification of an exact brain area used by the brain in a compensatory mechanism is important for each patient, in order to make the neuro-rehabilitation more effective. The disclosure positively identifies such brain areas in individual patients, and then frees the patient to engage in therapy outside of the rftMRI environment, for example in the home, using only fNIRS 200, a computer 700, and in various embodiments, FES 300 of some form.

System 100 can be incorporated into, for example, robotic devices, exoskeletons, prosthetic devices and other assistive devices. These devices can be useful to restore or improve mobility, and can also be used in the provision of therapeutic healthcare as a tool for treatment, or for measuring performance, especially in the field of motor rehabilitation. System 100 is further more portable and affordable as compared with EEG and fMRI assisting devices, and can therefore be used in a greater variety of applications, including, in particular, stroke rehabilitation. In addition, system 100 and methods of the disclosure simplify setting up the device for a new user as compared to other assisting devices.

System 100 can be used to restore normal or near normal function after stroke. The inventors have determined that stroke survivors can volitionally control the brain activation associated with movement preparation and execution. The inventors have additionally determined that statistically significant gains in motor control, and an ability to execute functional tasks, can be achieved in response to motor learning protocols with functional electric stimulation (FES). In accordance with the disclosure, use of fNIRS 200 in accordance with the disclosure to:

(1) decode the spatiotemporal patterns of hemodynamic brain signals pertaining to upper limb movements, namely, wrist flexion and extension;

(2) train stroke survivors with upper limb disability to enhance the self-regulation of these neural circuits with neurofeedback using system 100 in combination with contingent stimulation of wrist flexor and extensor muscles by functional electric stimulation (FES); and

(3) to evaluate the effects of this intervention to brain functional and structural connectivity, and clinical measures of motor coordination and quality of life in the patients. Collectively, the disclosure establishes neurofeedback training as both a basic research mechanism for elucidating the spatiotemporal hemodynamic patterns of movement (i.e., wrist flexion and extension), and and as an assessment and treatment tool for stroke patients with movement disorders.

In contrast to EEG and rtfMRI, the use of fNIRS 200 of the disclosure represents a brain imaging method with many benefits, including non-invasive imaging; greater speed and ease of setup (no gel, as in EEG-based BCI); lack of interference from movement artifacts as occurs in rtfMRI; portability during motor task performance in lab and clinic; and affordability in comparison to fMRI. More particularly, system 100 conditions spatiotemporal patterns of the brain responses associated with a desired movement, using fNIRS 200, concurrently with the afferent sensory input induced by peripheral stimulation using FES 30 of the muscles of the limbs that produces movement. Accordingly, use of fNIRS 200 in system 100 of the disclosure is as an rtfNIRS device.

The disclosure provides an fNIRS-BCI for motor rehabilitation based on real-time brain state classification. In healthy individuals, it is possible, using system 100, to develop an fNIRS brain pattern-model of joint movement, for example normal wrist flexion and extension; and based on the model distinguish and feedback, in real-time, fNIRS patterns of normal wrist flexion and extension from that of abnormal wrist flexion and extension.

Additionally, system 100 can be used for Motor Learning intervention with stroke survivors, and in particular, use fNIRS 200 for a series of brain signal training sessions in combination with the prior-tested interventional protocols with FES Motor Learning (FES ML; Daly et al 2010). Stroke survivors can then learn to modulate their brain patterns pertaining to wrist flexion/extension by repeated training with system 100.

Further in accordance with the disclosure, functional and structural changes in the brain and in patient behavior are quantified by a battery of measures taken pre-, midway- and post-intervention: functional connectivity changes, in an embodiment using fMRI 400 functional connectivity analysis; structural white matter connectivity changes (using diffusion tensor imaging (DTI) analysis); and clinical measures of motor coordination and quality of life.

The use of system 100 can further lead to functional connectivity enhancements among supplementary motor area, premotor area and primary motor areas. This can lead to improvements in the clinical measures of motor coordination and quality of life in stroke patients, for example. System 100 can be used with various motor learning strategies, including for example strategies based on the hypothesis that by training patients to produce normal brain activity one can influence brain plasticity that results in more normal brain function and motor behavior. Without being bound to a particular theory, the rationale for this approach is derived from extensive evidence from animal and human research showing that appropriate instrumental training regimens can change brain signal features of EEG, ECoG, single neuron, fMRI and fNIRS signals (Birbaumer et al., 2008, Fetz et al., 2007, Daly and Wolpaw 2008, Sitaram et al., 2009, Caria, Sitaram et al., 2011). Motor recovery after stroke is reported to be associated with structural and functional changes, such as neurite outgrowth in the peri-legional regions (Ng et al., 1988), increased synaptogenesis (Stroemer et al., 1995), increased axonal sprouting (Carmichael et al., 2001), and increased excitability (Scheine et al., 1996) of neurons.

Another strategy that can be used with system 100 is a motor learning strategy which use brain self-regulation to activate an external device that induces or assists in executing the desired movement. More particularly, improved motor function and sensory input that is contingent upon neural activity can induce brain plasticity and can lead to restoration of normal motor control. Without being bound to a particular theory, this strategy is supported by evidence that practicing movements that are as close to normal as possible might help to improve motor function (Nudo et al., 1996), by guiding newly sprouting axons to the appropriate cortical regions (Carmichael et al., 2002). For example, assistance of movement by FES through surface electrodes can improve upper limb function in individuals who have been impaired by stroke (Daly et al., 2005, Ring et al., 2005).

FIG. 1 illustrates a closed-loop fNIRS 200 BCI in combination with FES 300 of the disclosure. Brain signals are used to activate an FES device 300 that delivers an electric stimulus to, for example, index finger extensor muscles. The inventors have observed in one study that after nine training sessions there was improvement in volitional control of finger extension, showing that the BCI strategy of the disclosure using FES 300 can promote motor recovery, brain plasticity and motor recovery.

Again without begin bound to a particular theory, the disclosure is directed to promoting neuroplastic changes toward motor recovery, which is known as operant (instrumental) conditioning (Silvoni et al., 2011). It is based on the contingency of coupling a response and a reward/feedback. In the use of system 100, for a specific task, for example wrist extension, the spatiotemporal pattern of movement execution of wrist extension, and FES stimulation for extension are integrated to aid the intended motor action: for example to increase the amplitude and coordination of extension. Thus, FES 300 stimulation can be used to improve an existing but imperfect movement.

As a consequence of brain lesion after stroke, normal brain activation sometimes does not occur. The use of system 100 of the disclosure facilitates the activation of desired spatiotemporal brain activation patterns normally controlling a particular movement. This is followed by a reinforcing stimulus: the proprioceptive afferent perception induced by the FES stimulation of the wrist extensors. Based on operant conditioning theory, the disclosure defines the spatiotemporal pattern as the response (R), the FES stimulation as the discriminative stimulus (DS), and the afferent feedback coming from the FES stimulation as the reinforcing stimulus (RS). The conditioning procedure can be described as follows: repeatedly associating the spatiotemporal pattern of brain activity (R) to the proprioceptive afferent perception (RS) via the FES stimulation (DS) increases the probability of excitation of the correct/normal patterns of brain activation, leading to the facilitation of functional recovery.

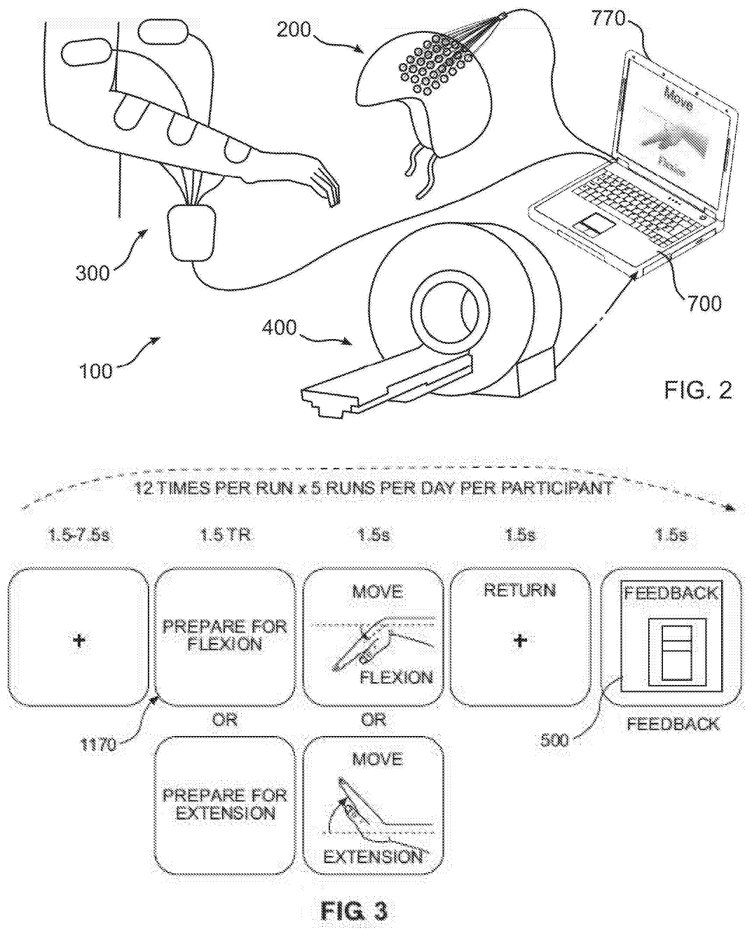

Referring to FIGS. 1 and 2, using portable fNIRS 200, signals from the brain are acquired, using in an embodiment a signal generator, photo multiplier, amplifier, and analog to digital converter. The output from fNIRS 200 are derived oxygenated hemoglobin (HbO) and deoxygenated-hemoglobin (HbR) concentrations corresponding to locations in the brain, as encoded in an electronic signal. These signals are transferred to a processor, for example an embedded system or a general purpose computer 700, which applies filtering, temporal smoothing, and artifact correction. These address particular instrument characteristics, as well noise factors such as head movement, probe movement, and physiological noise. As a result, the HbO and HbR values are correlated to particular feature vectors, or aspects of brain activity. Next, training, validation, and online classification takes place, as described elsewhere herein.

When a targeted area of the patient's brain becomes active, the area associated with a desired movement, an appropriate signal is sent to a portable FES 300, which aids the patient in carrying out the intended movement. Where possible, it is desired to eventually build and strengthen a signal from the brain, possibly to the point where FES assistance is not needed, although this may not always be possible. Ultimately, however, system 100 improves the issuance of a desired signal from the brain corresponding to the intended movement.

In accordance with the disclosure, healthy individuals first perform studies using system 100 in order to create spatiotemporal pattern models of normal wrist flexion and extension from fNIRS signals, in an fNIRS neuroimaging process. In the example of wrist flexion and extension, healthy subjects attempt to perform the desired movements in response to visual cues, which can be displayed on computer display 770, for example.

Following this, patients undergo a combined intervention of fNIRS 200 and FES 300 motor learning. For example, a stroke survivor with unilateral upper extremity disability due to stroke, can first undergo real-time training with system 100, for 3 days, each day consisting of 2 hours of fNIRS sessions, to train the control of neural patterns pertaining to wrist flexion and extension and to map all the brain regions involved in the task. System 100 will provide positive feedback on those trials when patients produce closer to normal wrist flexion or extension, as determined by the model-classifier generated from the healthy peoples' fNIRS signals. Patients further receive negative feedback for abnormal brain patterns associated with abnormal wrist flexion and extension, as compared with the healthy subjects.

With reference to FIG. 3, visual feedback is provided, in one embodiment, in the form of a graphically animated thermometer 500, which can be displayed upon display 770, in which positive and negative feedback can be represented by increasing and decreasing bars proportional to the online classification accuracies of the brain state classifier. In this particular aspect, methods can be used as disclosed in Robinson, N. Zaidi, A., Rana, M., Vinod A Prasad, Guan Cuntai, Niels Birbaumer, Sitaram, R. Real-time subject-independent pattern classification of overt and covert movements from fNIRS signals. Submitted to PLOS One.

In an embodiment, in a subsequent step, patients can also undergo fNIRS 200 training with contingent FES 300 stimulation for 10 more days to consolidate and establish the brain and behavioral effects of the fNIRS-FES combination of the disclosure. For wrist flexion therapy, FES 300 feedback will involve the online stimulation of the wrist muscles of the participant, whenever correct brain activation patterns are generated for the task of wrist flexion and extension, as determined by the pattern classifier. For other joints, an appropriate stimulation and feedback is provided for other muscles. The effects of training of the disclosure on the brain and on patient behavior is evaluated by performing pre- and post-treatment evaluations of functional and structural changes in the brain, and clinical measurements of movement recovery and quality of life.

FNIRS Feature Extraction and SVM Classification

Multi-channel temporal information of changes in concentration levels of blood oxy and de-oxy hemoglobin is used to classify wrist flexion and extension, or other movement as appropriate. The informative features from fNIRS recordings are extracted from the time averages of changes in oxyHB and deoxyHB concentration from the various channels located over the motor cortex. To perform classification, signal features at every unit time of movement preparation and execution (1.5 s) is considered, as shown in FIG. 3. A feature selection technique based on Mutual Information selects important features that correlate with the tasks from the input signals. This technique effectively chooses the channels that provide optimal discriminating information for the task performed by the patient. The feature set so selected for each time point is fed as input to the classifier for a SVM classification. If the classification accuracy at single participant levels prove to be robust and reliable, a follow-up classification at the group level will be performed. The group classifier will then represent the correct (normal) model of wrist extension and flexion, and hence will be used in real-time neurofeedback training of stroke patients.

BCI Neurofeedback Training of Normal Flexion & Extension in Stroke Patients

The development of an interventional therapy in accordance with the disclosure is conducted in 4 distinct steps, in chronological order, consisting of:

1) pretest of functional and structural measures of the brain using fMRI and DTI, and behavioral measures using EMG;

2) mid-test brain and behavioral measure (identical to pre-test);

3) fNIRS-FES neurofeedback stimulation training; and

4) post-test of brain and behavioral measures (identical to the previous tests).

The pre-, mid- and post-tests are planned so as to statistically evaluate the effects of neurofeedback training at three stages of the intervention. Comparisons in these measures will be made between patient groups. In the following paragraphs, we will elaborate on each step.

Step 1. Pre-Test of Brain Functional & Structural Images and Motor Performance

The pre-test first assesses functional activity and motor function, using simultaneous fMRI and EMG when patients perform wrist flexion and extension prior to neurofeedback training. In addition, resting state fMRI (RS-fMRI) will be performed to measure brain activation during resting state in patients and to investigate how neurofeedback training might change these activations. In addition to providing a baseline condition, the pre-test provides the information necessary to evaluate brain activity in ROIs responsible for motor function, namely, SMA, M1, premotor cortex and parietal cortex. EMG signals will be analysed using standard EMG analysis procedure as well as pattern classification between wrist flexion and extension. In patients, MR structural measurements, white matter fibre tractography using MR diffuse tensor imaging (DTI; Varkuti, Sitaram et al., 2011), and cerebral blood flow using MR arterial spin labeling (ASL) sequences will be performed. DTI and ASL measurements and analysis in the pretest will act as measures of structural baseline in patients, and when compared with intermediate and post-test measures, will allow us to evaluate neuroplastic changes, in terms of, structural connectivity and blood perfusion changes.

Step 2. Neurofeedback Protocol.

With reference to FIG. 3, the neurofeedback training protocol is based on the experimental protocol for performing functional imaging of wrist flexion and extension with an important distinction that at the end of each wrist extension or flexion movement a visual feedback in the form of a graphical thermometer will be provided to the participant for 1.5 s. The feedback is a representation, in terms of the increase in the number of bars of the thermometer, how successful the patient was in producing brain activation patterns similar to the normal brain patterns (derived from healthy subjects) pertaining to the specific movement, for example, wrist flexion or extension. The greater the similarity, the greater will be the number of bars in the thermometer. The number of bars are normalized to the full range of SVM output values; the baseline being the average SVM value, the minimum number bars will represent the maximum negative value (for class −1), and maximum number of bars pertain to the maximum positive value (for class 1) of the SVM.

FNIRS-BCI Feedback Training Protocol.

Step 3. Intermediate-Test of Brain Functional & Structural Images and Motor Performance.

The intermediate tests are identical to the pretests in both the brain and behavioral measures. Differences in brain functional and structural connectivity, and behavioral measures, are mainly expected to take place between the pre- and post-tests. The mid-test provides information regarding how and where in the brain these changes occur.

Step 4. Real-Time fNIRS-FES-BCI Neurofeedback-Stimulation Training

Acquired signals from fNIRS 200 are received online in the BCI-processing computer 700 that consists of the following realtime modules: feature extraction, selection, and classifier. For every time point of the neurofeedback protocol, system 100 classifies the fNIRS signals into wrist flexion or extension. Depending on whether a normal wrist flexion or extension was detected, the classifier's output is translated into a command to the FES system to stimulate the wrist flexors and extensors in an appropriate FES paradigm. The disclosure can be carried out with any of a variety of FES 300 devices. In one embodiment, the Motionstim 8 stimulator from MEDEL GmbH, Hamburg, Germany is used, in which, again referring to the example of wrist movement, two unipolar electrodes of oval shape (4×6 cm) are placed on the extensor digitorum communis (EDC) and two electrodes were placed on the flexor digitorum communis (FDC) of the right forearm following physical landmarks. The pulse width is fixed to 300 micro-s. The stimulation frequency can be changed to either 20 Hz or 30 Hz. The amplitude of stimulation is adjusted for each individual to cross the motor threshold of both muscles to produce finger extension and flexion. Average amplitude of stimulation for the EDC is 19.9+/−3.8 mA and for the FDC was 17.5+/−3.6 mA. Patients are instructed to let their hands be moved by the stimulation.

Step 5. Post-Test of Brain Functional & Structural Images and Motor Performance

The post-tests are identical to the intermediate tests in terms of both the brain and behavioral measures.

6. Possible Discomforts and Risks

A variety of fNIRS devices can be used to carry out the disclosure. In an embodiment, model ETG-400, of (Hitachi Medical Systems Europe is used, which is a high-quality, real-time cerebral-cortex-imaging and measurement device. This highly portable, bedside optical topography system can be used to inspect and measure live, in-vivo images of the human brain, while it is working. It captures and measures hemoglobin levels, while the brain functions. This system is non-invasive and requires little or no patient restraint. In particular, this state-of-the-art optical topography system transmits near-infrared light into the patient's head and collects the reflected information from the cerebral cortex. Functional Near Infrared Spectroscopy (fNIRS) is a non-invasive optical brain imaging technique that determines relative levels of oxygenated (oxyHb) and deoxygenated hemoglobin (deoxy-Hb) based on the absorption characteristics of near infrared light for different form of hemoglobin. fNIRS allows for the acquisition of information about the cerebral blood flow. The ETG-4000 system employs laser diodes in the wavelength range of about 690-900 nm. The system uses class 1M laser products in accordance with IEC (International Electrochemical Commission) guideline IEC825. Class 1M laser products are considered safe based on current medical knowledge.

fNIRS uses near-infrared spectroscopy and allows for indirect (i.e., non-invasive) measurement of brain activation. For this particular device, a plastic hood that holds the sensors is placed with elastic bands around the participants head. The planned measurements will be done with a clinically approved device that has not been associated with any known side-effects or safety risks. This fNIRS system can be comfortably worn for 60 minutes.

Patients are monitored very carefully during the entire fMRI or fNIRS procedures, and repeatedly checked to ensure comfort. At the end of each visit, patients can be asked to respond to a questionnaire to learn of any possible discomfort related to fMRI and fNIRS scanning.

Functional Electrical Stimulation (FES): For the Motionstim 8 stimulator, small electrodes are placed over the muscle to be stimulated and wires connect these electrodes to the stimulator. The electrical stimulation is set to the smallest current required for the muscle to contract. The stimulation causes a tingling sensation on the skin, which might be uncomfortable but it is virtually painless. Occasionally the stimulation might cause irritation of the skin, which can be easily addresses by changing the level of current or by changing the electrodes.

Using the foregoing procedure, stroke patients or others with brain impairment can learn to modulate their brain patterns pertaining to wrist flexion/extension or any other muscle or joint movement, by repeated training. The disclosure provides superior advantages in terms of portability and affordability and thus more freedom to develop various applications, as compared to robotic devices, prosthetics, exoskeletons and assistive devices using EEG or fMRI. In terms of the features provided, the disclosure is lower in expenses as compared to an fMRI-BCI. Further, the disclosure increases productivity as compared to any of the existing devices because of its compact and portable dimensions. System 100 provides a simpler setup process, with a shorter setup time, as compared to other BCI devices.

The disclosure provides a real-time method for subject-specific and subject-independent classification of multichannel fNIRS signals using support-vector machines (SVM), and demonstrates its utility as an online neurofeedback system. The inventors used left versus right hand movement execution and movement imagery as study paradigms in a series of experiments. In the first two experiments, activations in the motor cortex during movement execution and movement imagery were used to develop subject-dependent models that obtained high classification accuracies thereby indicating the robustness of our classification method. In the third experiment, a generalized classifier-model was developed from the first two experimental data, which was then applied for subject-independent neurofeedback training. Application of this method in new participants showed mean classification accuracy of 63% for movement imagery tasks and 80% for movement execution tasks. These results, and their corresponding offline analysis demonstrate that SVM based realtime subject-independent classification of fNIRS signals is feasible. This method has important applications in the field of hemodynamic BCIs, and neuro-rehabilitation where patients can be trained to learn spatio-temporal patterns of healthy brain activity.

Protocol

To analyze brain activations during bilateral hand movement execution (ME) and imagery (MI), the experimental protocol was designed to consist of five conditions, namely movement execution of left and right hand, motor imagery of left and right hand, and rest condition. The participants were asked to perform repetitive hand movements similar to clenching and unclenching an imaginary ball at a frequency of −1 Hz during motor execution. During movement imagery, participants were asked to imagine similar movements, without actually moving their hands. No physical movement was observed in any subject during the imagery tasks.

Participants were asked to participate in five runs of one experiment, each of which consisted of six task blocks separated by seven rest blocks. In FIG. 6, Left and Right refers to left and right hand's movement for both execution and imagery tasks. Each participant was seated in front of the screen that displayed the visual cues. As per the protocol, the cues for each block were as follows: a blue screen with a black dot for “Rest”, a red screen with a Right arrow, for “Task—Right” and green screen with Left arrow, for “Task—Left”. An activation-level meter (hereafter called thermometer as it is depicted graphically as a thermometer) with baseline level indicated at its middle, appeared on center of the screen during the training runs.

For test runs, neurofeedback was given as the thermometer grades. The dynamic range of the thermometer was 20 units or levels. Our study comprises three different experiment paradigms as shown in Table 1. In all the experiments, the initial one or two runs were used for training the system. No feedback was provided during training runs, and the thermometer grade remained at the baseline. Following this, the subjects were instructed to perform the test runs with neurofeedback. Feedback was provided as increase or decrease in thermometer grade during correct and incorrect classification respectively.

TABLE 1
Overview of experimental design for various experiments

| | Run 1 | Run 2 | Run 3 | Run 4 | Run 5 |
|---|---|---|---|---|---|
| Experiment 1 | ME^{a} | ME | ME | MI^{b} | MI |
| Experiment 2 | MI | MI | MI | MI | ME |
| Experiment 3 | ME | MI | MI | MI | ME |

^{a}ME represents Motor execution
^{b}MI represents Motor Imagery.
Italics indicates runs used for training the classifier.

Experiment 1.

An objective of this study was to implement real-time subject-dependent classification of bilateral hand movement using a movement execution trained BCI. The system was validated on bilateral motor execution and imagination data, to provide realtime classification results and spatial activation patterns for further analysis. In the experiment, the classifiers were adapted as per Eq (9) for the following test runs. Run 3 tested the classifier on ME for each subject. Runs 4 and 5 were used to test classification of MI based on ME models, and the subjects were asked to imagine the movements. In all the test runs, the subjects were provided a visual feedback based on the classification output.

Experiment 2.

An objective of this study was to perform a corollary to Experiment 1, i.e., to implement real-time subject-dependent classification and feedback of left versus right hand motor imagery, based on a classifier built using covert hand movement data. In the MI runs, 1 to 4, the subjects were instructed to imagine the movement they had practiced. The first run was used for training the classifier. For Runs 2 to 4, the classifier was updated after each Run, as per Eq (9). The performance of subjects performing MI was tested using the classifier and a neurofeedback was provided. Run 5 was used to test classification of ME based on MI models (with the classifier modeled based on the last two MI runs) and the subjects were asked to perform ME.

Experiment 3.

An objective was to demonstrate the feasibility of a Subject-Independent Classifier (SIC) built from the ensemble data of all participants from Experiment 1, performing hand movement execution. At the beginning of this experiment, a practice session was provided where the subjects were asked to perform hand clenching actions. During the experiment, in test runs 2, 3 and 4, the subjects were asked to perform MI of the practiced movements without moving their hands. In the ME run 5, the subjects were asked to execute the movement. Real-time classifications of overt and covert movements from new subjects were performed using the SIC and neurofeedback was provided in all the runs.

Feature Extraction and Selection

In a study, the inventors used multi-channel temporal information of changes in concentration levels of blood oxy hemoglobin (HbO) to classify volitional overt and covert hand movements. The discriminative features from fNIRS recordings are extracted from the time averages of changes in HbO concentration from the various channels located over the motor cortex. The real-time classification of signal features and estimation of neurofeedback are performed at every unit time (1 second). Hence, for an Nₜ-channel arrangement, the features extracted at k^{th} second of a trial from n^{th} channel is given by,

$${f}^{n} ( k ) = {\sum }_{i ∈ I to {f}_{s}} Δ {HbO}_{i}^{n} ( k ) \qquad [ 1 ]$$

where fs is the sampling frequency and n=1 to Nₜ. The feature set at k^{th} instant is given by,

F(k)={f¹(k),f²(k),f³(k), . . . ,f^{N}^{ₜ}(k)} [2]

A feature selection technique based on mutual information selects N<Nt features from Eq (2). This technique effectively chooses the channels that provide optimal discriminating information for the task performed by the participant. For an Nₜ-dimensional feature set F, the mutual information based technique selects, S⊂F, an N-dimensional subset that maximizes the mutual information, I(F;ω), where ω represents each class i∈{1,2}. Mutual information is given by,

$$\begin{matrix}I ( F ; ω ) = H ( ω ) - H ( ω \vert F ) , ω ∈ \{ {ω}_{1} , {ω}_{2} \} & [ 3 ] \\ H ( ω \vert F ) = - {\sum }_{i = \{ 1 , 2 \}} p ( {ω}_{i} \vert F ) {log}_{2} p ( {ω}_{i} \vert F ) & [ 4 ]\end{matrix}$$

where, H(ω) denotes the class entropy and H(ω|F) gives the conditional entropy. The conditional probability p(ω|F) is estimated using Parzen window method. The mutual information for all the Nt features are calculated and the best N features are selected to obtain,

S(k)={fⁿ(k)}, n∈selected N features [5]

The value of N is set to 12 in this work, and it is ensured that equal number of features are selected from both left and right hemispheres. The performance of the system may vary depending on N. The feature set S(k) for every k^{th} instant is fed as input to the classifier for realtime classification and to calculate neurofeedback.

The figures present an overview of the real-time neuro-feedback binary classification system of the disclosure. FIG. 6 presents an experiment protocol and timeline for the experiment: Runs 1-5 are separated by 5-10 minutes rest periods. The sequence of blocks with their durations for each Run is shown under Run 1.

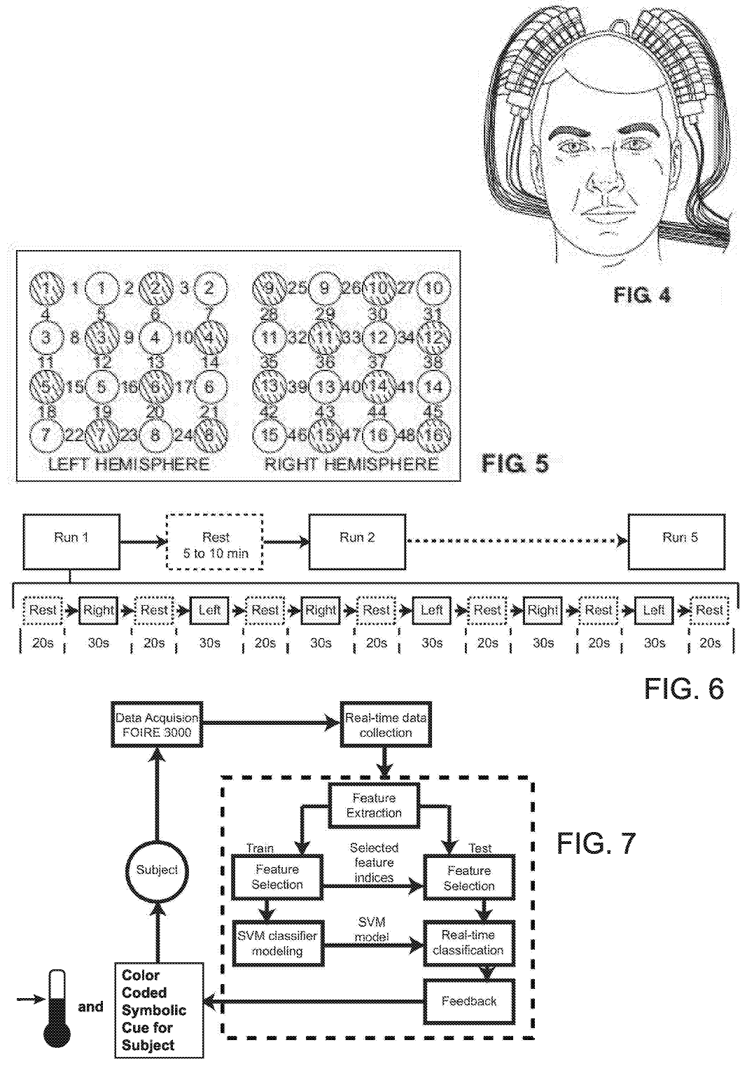

FIG. 4 diagrammatically illustrates an arrangement of optodes and the headmount. The optodes are placed over the motor area and are arranged in a 4×4 checkerboard topography. This topography is reflected in FIG. 5, in which the cross-hatched and white circles indicate emitters and detectors respectively. The numbers 1-48 indicate the recorded channels.

The architecture of the designed system is depicted in FIG. 7, indicating its various functional units. The feedback generated by BCI is displayed to the subject as indicated, and as described elsewhere herein.

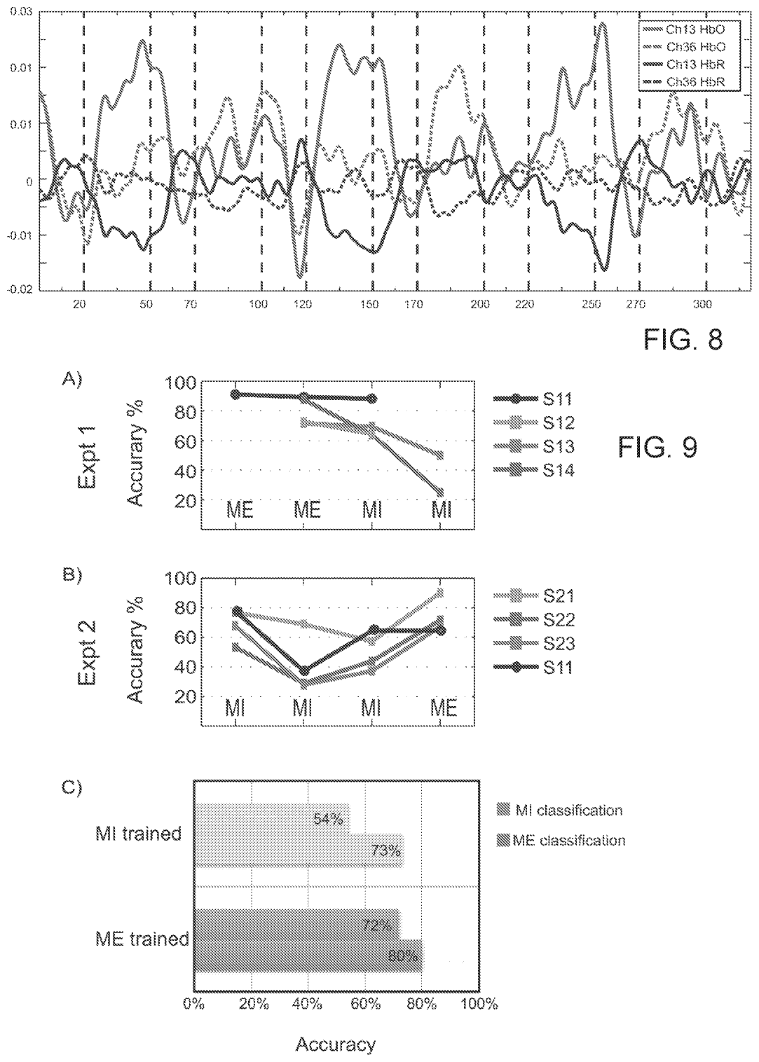

FIG. 8 presents a sample time course of activations during motor execution. The pre-processed data from Experiment 1, Subject S11, Run 3 is shown. Channel 13 and 36 are from PMC in left and right hemispheres. The contralateral activations of HbO and a dip in HbR can be clearly identified from the plots.

Support Vector Machines (SVM)

SVM is a supervised learning technique that creates a boundary between two classes of data based on a set of available training samples. It designs a decision function that optimally separates the two classes in the training data. In this study we use a linear-SVM to separate left versus right hand movements. For real time classification, we consider the features obtained at each instant k as a separate training data sample. The data sample at k^{th} instant is the feature vector denoted by S(k) or S^{k}. The SVM-classifier determines a weight vector W, that discriminates a class against the other by the projection W'S and linear discriminant rule,

$$ω \{ \begin{matrix}∈ {ω}_{i} & {W}^{′} {S}^{k} \geq b \\ ∈ {ω}_{i} & {W}^{′} {S}^{k} < b\end{matrix} \qquad [ 6 ]$$

where b is a bias. This vector is determined by minimizing the cost function,

J(W)=½∥W∥² [7]

subject to the constraint,

Y^{k}(W′·S^{k}−b)≥b, k=1 to K [8]

where Y^{k} is the class label corresponding to S^{k}, that is a sample from the training data set {S¹, S², . . . , S^{K}} and K is the number of training data samples. The SVM classifier thus modeled is used to classify or to determine the label of incoming data samples.

Adapting Classifier and Feature Selector

For neurofeedback training applications, the BCI of the disclosure is designed to provide feedback information regarding the quality of the performed task to the user in real time. Considering the non-stationarity of the neural signals there is a need to adaptively update the classifier and feature selector in the system.

In the subject-dependent classifier experiments, the initial run is used to select the most informative features and model the SVM classifier that optimally discriminates the binary class data. This is used to classify the data samples of Run 2 in real time. As given in (9), from the 3^{rd} Run onwards, the classifier is re-modeled using the data from two previous runs.

$${\{ N ; W \}}^{( r )} = \{ \begin{matrix}C ( {S}^{( r - 1 )} ) , & r = 2 \\ C ( \vert {S}^{( r - 1 )} ; {S}^{( r - 2 )} \vert ) , & r > 2\end{matrix} \} \qquad [ 9 ]$$

where, r is the run number, S^{(r)} is the data set collected during Run r and C denotes the feature selection and classifier modeling functions. Moreover, a bias cancellation is performed from Run 2 onwards that subtracts the average of SVM output during the Rest block from the following Task block. The real-time system thus adopts a between-runs adaptive strategy of retraining classifiers after each run and within-run adaptive bias correction of SVM outputs.

Participants

The data were recorded from 11 healthy participants (both male and female, aged 21-35). All participants signed a written informed consent. The study was approved by the Institutional Review Board, Faculty of Medicine of the University of Tuebingen, Germany. Each participant was compensated monetarily for participation in the experiment.

Data Acquisition

FNIRS signals were acquired using a Shimadzu FOIRE-3000 imaging system operating at a sampling rate of 7.69 Hz, using wavelengths of 780 nm and 830 nm from laser sources. Emitters and detectors were separated by 25 mm, and were placed on top of the participant's head using a semi-flexible head mount. Sixteen sources and detectors were arranged in two 4-by-4 checkerboard topographies, as shown in FIG. 4 centered on C3 and C4 of the International 10-20 System. This arrangement covered most of the primary motor, pre-motor and somatosensory cortices.

Real-Time fNIRS-BCI System Schematic

The architecture of the real-time system designed is shown in FIG. 7. FNIRS signals are received online in the BCI-processing computer from the FOIRE-3000 equipment. The BCI processing system consists of a feature extractor, a feature selector and a classifier. The data are fed into the processing system in real-time. As described above, we extract the relevant features from the recorded fNIRS data. The data from training runs are used to select the informative features and to model the classifier as explained above. For the test runs, after the movement task stimulus onset, a bias correction is performed and the extracted features are classified in real-time using the SVM model created. The classified output is generated at every second so as to provide feedback in real time. This output is presented to the participant in the form of a graphical thermometer in which a correct classification would lead to a unit rise in the thermometer, and incorrect classifications would lead to a unit fall in the thermometer reading. The thermometer reading remains at 0 (middle) during “Rest” period and returns to this position at the end of every movement task.

Offline Data Analysis

The preprocessing steps used to improve the Signal-to-Noise ratio and derive optimal information from recorded fNIRS data were as follows: the data was baseline corrected followed by pre-coloring using a hemodynamic response function-low pass filter; the global trends were removed using Wavelet-Minimum Description Length technique. A sample time course of activation of pre-processed fNIRS recording is shown in FIG. 8, which plots the HbO and HbR signals from channels 13 and 36 (corresponding to primary motor cortex (BA4) in left and right hemispheres respectively) from Subject S11, Experiment 1, Run 2 From the figure, distinct changes can be seen in the contralateral activity of oxy- and deoxy-hemoglobin concentrations. These changes were utilized for feature extraction and modeling of the SVM-based classifier. To ensure stationarity of the training data used to create classifiers, 5-fold cross-validation analysis was performed. The training data was randomly split into five subsets. In each crossvalidation fold, data from four subsets were used to select features and model classifier that was used to classify the remaining test subset. The process was repeated to test all the subsets and an average performance over all the folds was calculated. The low values of training classification accuracy's standard deviations indicated the low variance of the training dataset used (not shown). fNIRS signals were also analyzed to determine statistically significant spatial activations by a univariate approach using SPM 5 fNIRS toolbox. The spatial plots of mutual information obtained from Eq (4) and the SVM outputs obtained from Eq (8) are also reported among the various results.

Real-Time Classification

The motor performance of subjects is evaluated in real-time by online feature extraction and SVM classification of bilateral motor tasks and the percentage classification accuracies are reported. FIG. 2 summarizes the performance of the proposed real-time classification system for overt movement execution and imagery with neurofeedback. The results indicated are percentage classification accuracies attained by subjects in each of the runs for various tasks indicated using MI (motor imagery) and ME (motor execution) labels. To comply with experimental guidelines, subjects were allowed to discontinue the experiment if they experienced fatigue. The Experiments 1 and 2 used subject-dependent classifier models for bilateral MI and ME classification. In Experiment 1, the average classification accuracy over four subjects obtained for runs 3 and 4 are 80% (ME) and 72% (MI) respectively, where the task performed is indicated within brackets. Not all subjects were able to complete the five runs due to fatigue. In Experiment 2, for all subjects the accuracy falls after the first run and improves afterwards. On an average, the classification accuracies are reported as 69% (MI), 41% (MI), 51% (MI) and 73% (ME) for runs 2, 3, 4 and 5 respectively. The last run (run 5) used the classifier trained on MI for online classification of bilateral ME. A general trend seen in the results is a dip in performance after the first run, followed by gradual rise. Although the paradigm we use is insufficient to prove the effect of neurofeedback training and its learning effect in subjects, the performance trend obtained indicates subject's capability to identify and enhance motor control strategy after each run. Longer experiment sessions might reveal more information on such a learning curve. The simple adaptive strategies of re-training classifier and bias correction seem to work efficiently in this real-time system.

FIG. 9 presents real-time classification performance for experiments 1 and 2. FIGS. 9A and 9B present the percentage classification accuracies for online binary (right v/s left motor tasks) classification for 7 subjects for Experiment 1 (A) and Experiment 2(B). The motor tasks involved are right and left Motor Execution (ME) and Motor Imagery (MI). Note: Subject S11 was common between both experiments 1 and 2. FIG. 9C presents a comparison of mean classification accuracies of motor imagery and execution when classifier was trained on either motor imagery or motor execution.

The real-time neurofeedback system using the signal processing strategy of the disclosure offers at least the following advantages: (1) the system identifies the optimal discriminative features based on mutual information and applies these for classifier modeling, (2) the classifier adapts by itself after each run, making use of the data collected in the previous run, and (3) the bias correction within runs compensates for the dc shift in the feature space to provide better classification performance. The channels chosen using mutual information based feature selection are found to lie over the motor cortex in most of the cases. The bias correction that provides an intra-run classifier adaptation clearly results in better classification accuracies. The performance of the binary classifiers used in various runs is demonstrated using ROC curves, with the operating point defining the threshold at which the system uses the classifier model. The runs with good classification accuracies generate almost ideal ROC curves, with their operating point in the high TPR-low FPR region. Each of the parameters are inter-related, and together, they define the real-time system.

This study and the devices and methods of the disclosure are expected to bring researchers and health practitioners closer to assist stroke-patient rehabilitation using the subject-independent classifier with real-time neurofeedback. The study in healthy subjects can be applied to patients with more optimizations. Also, the subject-independent motor activation patterns from healthy subjects can be used to train patients with motor disabilities to imitate and later even generate similar patterns.

In prior studies, normal motor control and function were not completely attained, and not all subjects had significant improvement. The disclosure carries out a novel approach of closed-loop BCI for effective recovery of movement by conditioning spatio-temporal patterns of the brain responses associated with the desired movement, concurrently with the afferent sensory input induced by peripheral stimulation of the muscles of the limbs that produces movement. The disclosure demonstrates, with real-time subject-independent classification and feedback, that by training patients to produce normal brain activity, one may be able to influence brain plasticity that results in normal brain function and motor behavior. In the case of motor function, this strategy is supported by evidence that practicing movements that are as close to normal as possible might help to improve motor function, by guiding newly sprouting axons to the appropriate cortical regions. The development of a subject-independent classifier based BCI is thus an important step towards successful stroke rehabilitation.

The disclosure includes a focus on real-time binary classification of left versus right hand movement execution and imagery using an SVM based classifier. A subject-independent pattern classifier generated from movement execution data using the feature extraction and selection strategy discussed above was used in real-time classification of MI and ME. The neuronal activity correlates between MI and ME were explored and utilized to create a generic classifier. The performance of the system in terms of bilateral movement classification accuracies obtained in various sessions of the different subjects are reported. The classifier parameters obtained in each of the experiments conducted, indicating robust and accurate performance, are separately discussed. The data are analyzed offline to identify the spatial and temporal activations and the results were also demonstrated. The results are promising, and demonstrate that the disclosure can be used in a real-time BCI system for clinical rehabilitation purposes.

Additional details can be found in Robinson N, Zaidi A D, Rana M, Prasad V A, Guan C, Birbaumer N, et al. (2016) Real-Time Subject-Independent Pattern Classification of Overt and Covert Movements from fNIRS Signals. PLoS ONE 11(7): e0159959. doi:10.1371/journal. pone.0159959, the contents of which are incorporated herein by reference.

Example Computing System

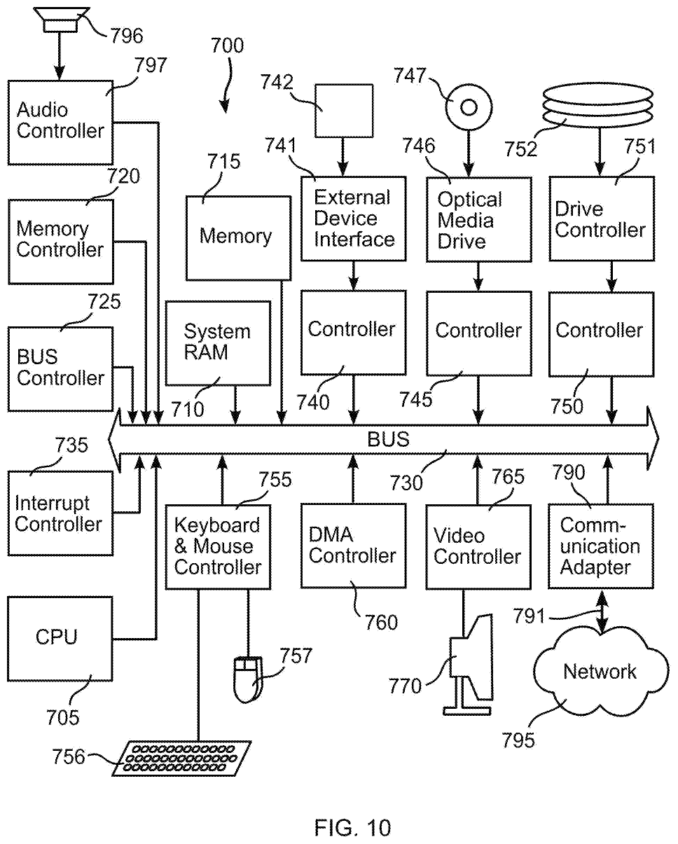

FIG. 10 illustrates the system architecture for a computer system 700, such as a process controller, or other processor on which or with which the disclosure may be implemented. The exemplary computer system of FIG. 10 is for descriptive purposes only. Although the description may refer to terms commonly used in describing particular computer systems, the description and concepts equally apply to other systems, including systems having architectures dissimilar to FIG. 10. Computer system 700 can control temperatures, electrodes, probes, power supplies, and other instruments, including through the use of actuators and transducers. One or more sensors, not shown, provide input to computer system 700, which executes software stored on non-volatile memory, the software configured to received inputs from sensors or from human interface devices, in calculations for controlling system 200.

Computer system 700 includes at least one central processing unit (CPU) 705, or server, which may be implemented with a conventional microprocessor, a random access memory (RAM) 710 for temporary storage of information, and a read only memory (ROM) 715 for permanent storage of information. A memory controller 720 is provided for controlling RAM 710.

A bus 730 interconnects the components of computer system 700. A bus controller 725 is provided for controlling bus 730. An interrupt controller 735 is used for receiving and processing various interrupt signals from the system components.

Mass storage may be provided by DVD ROM 747, or flash or rotating hard disk drive 752, for example. Data and software, including software 400 of the disclosure, may be exchanged with computer system 700 via removable media such as diskette, CD ROM, DVD, Blu Ray, or other optical media 747 connectable to an Optical Media Drive 746 and Controller 745. Alternatively, other media, including for example a media stick, for example a solid state USB drive, may be connected to an External Device Interface 741, and Controller 740. Additionally, another computing device can be connected to computer system 700 through External Device Interface 741, for example by a USB connector, BLUETOOTH connector, Infrared, or WiFi connector, although other modes of connection are known or may be hereinafter developed. A hard disk 752 is part of a fixed disk drive 751 which is connected to bus 730 by controller 750. It should be understood that other storage, peripheral, and computer processing means may be developed in the future, which may advantageously be used with the disclosure.

User input to computer system 700 may be provided by a number of devices. For example, a keyboard 756 and mouse 757 are connected to bus 730 by controller 755. An audio transducer 796, which may act as both a microphone and a speaker, is connected to bus 730 by audio controller 797, as illustrated. It will be obvious to those reasonably skilled in the art that other input devices, such as a pen and/or tablet, Personal Digital Assistant (PDA), mobile/cellular phone and other devices, may be connected to bus 730 and an appropriate controller and software, as required. DMA controller 760 is provided for performing direct memory access to RAM 710. A visual display is generated by video controller 765 which controls video display 770. Computer system 700 also includes a communications adapter 790 which allows the system to be interconnected to a local area network (LAN) or a wide area network (WAN), schematically illustrated by bus 791 and network 795.

Operation of computer system 700 is generally controlled and coordinated by operating system software, such as a Windows system, commercially available from Microsoft Corp., Redmond, Wash. The operating system controls allocation of system resources and performs tasks such as processing scheduling, memory management, networking, and I/O services, among other things. In particular, an operating system resident in system memory and running on CPU 705 coordinates the operation of the other elements of computer system 700. The present disclosure may be implemented with any number of commercially available operating systems.

One or more applications, such as an HTML page server, or a commercially available communication application, may execute under the control of the operating system, operable to convey information to a user.

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Accordingly, all expedient modifications readily attainable by one versed in the art from the disclosure set forth herein that are within the scope and spirit of the present disclosure are to be included as further embodiments of the present disclosure.

### REFERENCES

The following references are incorporated by reference herein in their entirety:
1. Sulzer J., Haller S., Scharnowski F., Weiskopf N., Birbaumer N., Blefari M. L., et al. (2013). Real-time fMRI neurofeedback: progress and challenges. Neuroimage, 76, 386-399. doi: 10.1016/j.neuroimage. 2013.03.033 PMID: 23541800
2. Birbaumer N, Ruiz S, Sitaram R (2013). Learned regulation of brain metabolism. Trends in Cognitive Sciences 17: 295-302. doi: 10.1016/j.tics.2013.04.009 PMID: 23664452
3. Sitaram R., Lee S., Ruiz S., Rana M., Veit R., & Birbaumer N. (2011). Real-time support vector classification and feedback of multiple emotional brain states. Neuroimage, 56(2), 753-765. doi: 10.1016/j. neuroimage.2010.08.007 PMID: 20692351
4. Sitaram R., Zhang H., Guan C., Thulasidas M., Hoshi Y., Ishikawa A., et al. (2007). Temporal classification of multichannel near-infrared spectroscopy signals of motor imagery for developing a brain-computer interface. NeuroImage, 34(4), 1416-1427. PMID: 17196832
5. Mihara M., Miyai I., Hattori N., Hatakenaka M., Yagura H., Kawano T., et al (2012). Neurofeedback using real-time near-infrared spectroscopy enhances motor imagery related cortical activation. PloS one, 7(3), e32234. doi: 10.1371/journal.pone.0032234 PMID: 22396753
6. Ang K. K., Chin Z. Y., Zhang H., & Guan C. (2012). Mutual information-based selection of optimal spatial-temporal patterns for single-trial EEG-based BCIs. Pattern Recognition, 45(6), 2137-2144.
7. Ang, K. K., Yu, J., & Guan, C. (2012, June). Extracting and selecting discriminative features from high density NIRS-based BCI for numerical cognition. In The 2012 International Joint Conference on Neural Networks (IJCNN) (pp. 1-6). IEEE.
8. Vapnik V. N., & Vapnik V. (1998). Statistical learning theory (Vol. 1). New York: Wiley.
9. Coyle S, Ward T, Markham C (2004) Physiological noise in near-infrared spectroscopy: implications for optical brain computer interfacing. Conf Proc IEEE Eng Med Biol Soc 6: 4540-4543. PMID: 17271316
10. Ye J. C., Tak S., Jang K. E., Jung J., & Jang J. (2009). NIRS-SPM: statistical parametric mapping for near-infrared spectroscopy. Neuroimage, 44(2), 428-447. doi: 0.1016/j.neuroimage.2008.08.036 PMID: 18848897
11. Abdelnour A. F., & Huppert T. (2009). Real-time imaging of human brain function by near-infrared spectroscopy using an adaptive general linear model. Neuroimage, 46(1), 133-143. doi: 10.1016/j. neuroimage.2009.01.033 PMID: 19457389
12. Dechent P., Merboldt K. D., & Frahm J. (2004). Is the human primary motor cortex involved in motor imagery?. Cognitive Brain Research, 19(2), 138-144. PMID: 15019710
13. Daly J. J., Hogan N., Perepezko E. M., & Krebs H. I. (2005). Response to upper-limb robotics and functional neuromuscular stimulation following stroke. Journal of rehabilitation research and development, 42(6), 723. PMID: 16680610
14. Daly J. J., Cheng R., Rogers J., Litinas K., Hrovat K., & Dohring M. (2009). Feasibility of a new application of noninvasive brain computer interface (BCI): a case study of training for recovery of volitional motor control after stroke. Journal of Neurologic Physical Therapy, 33(4), 203-211. doi: 10.1097/NPT. 0b013e3181c1fc0b PMID: 20208465
15. Silvoni S., Ramos-Murguialday A., Cavinato M., Volpato C., Cisotto G., Turolla A., et al. (2011). Braincomputer interface in stroke: a review of progress. Clinical EEG and Neuroscience, 42(4), 245-252. PMID: 22208122
16. Nudo R. J., Wise B. M., SiFuentes F., & Milliken G. W. (1996). Neural substrates for the effects of rehabilitative training on motor recovery after ischemic infarct. Science, 272(5269), 1791. PMID: 8650578
17. Carmichael S. T., Wei L., Rovainen C. M., & Woolsey T. A. (2001). New patterns of intracortical projections after focal cortical stroke. Neurobiology of disease, 8(5), 910-922. PMID: 11592858
18. ANG, K. K. et al. A clinical study of motor imagery-based brain-computer interface for upper limb robotic rehabilitation. Conf Proc IEEE Eng Med Biol Soc, v. 2009, p. 5981-4, 2009. ISSN 1557-170X (Print) 1557-170x.
19. BIRBAUMER, N.; MURGUIALDAY, A. R.; COHEN, L. Brain-computer interface in paralysis. Curr Opin Neurol, v. 21, n. 6, p. 634-8, December 2008. ISSN 1350-7540 (Print) 1350-7540.
20. CARMICHAEL, S. T.; CHESSELET, M. F. Synchronous neuronal activity is a signal for axonal sprouting after cortical lesions in the adult. J Neurosci, v. 22, n. 14, p. 6062-70, Jul. 15, 2002. ISSN 0270-6474.
21. DALY, J. J.; WOLPAW, J. R. Brain-computer interfaces in neurological rehabilitation. Lancet Neurol, v. 7, n. 11, p. 1032-43, November 2008. ISSN 1474-4422 (Print) 1474-4422.
22. DALY, J. J. et al. Recovery of coordinated gait: randomized controlled stroke trial of functional electrical stimulation (FES) versus no FES, with weight-supported treadmill and over-ground training. Neurorehabil Neural Repair, v. 25, n. 7, p. 588-96, September 2011. ISSN 1545-9683.
23. FETZ, E. E. Volitional control of neural activity: implications for brain-computer interfaces. J Physiol, v. 579, n. Pt 3, p. 571-9, Mar. 15, 2007. ISSN 0022-3751 (Print) 0022-3751.
24. LO, Y. L. et al. Cerebellar control of motor activation and cancellation in humans: an electrophysiological study. Cerebellum, v. 8, n. 3, p. 302-11, September 2009. ISSN 1473-4222.
25. RING, H.; ROSENTHAL, N. Controlled study of neuroprosthetic functional electrical stimulation in sub-acute post-stroke rehabilitation. J Rehabil Med, v. 37, n. 1, p. 32-6, January 2005. ISSN 1650-1977 (Print) 1650-1977.
26. SACCHET, M. D. et al. Volitional control of neuromagnetic coherence. Front Neurosci, v. 6, p. 189, 2012. ISSN 1662-453x.
27. SCHIENE, K. et al. Neuronal hyperexcitability and reduction of GABAA-receptor expression in the surround of cerebral photothrombosis. J Cereb Blood Flow Metab, v. 16, n. 5, p. 906-14, September 1996. ISSN 0271-678X (Print) 0271-678x.
28. SITARAM, R.; CARIA, A.; BIRBAUMER, N. Hemodynamic brain-computer interfaces for communication and rehabilitation. Neural Netw, v. 22, n. 9, p. 1320-8, November 2009. ISSN 0893-6080.
29. SITARAM, R. et al. FMRI brain-computer interface: a tool for neuroscientific research and treatment. Comput Intell Neurosci, p. 25487, 2007. ISSN 1687-5265 (Print).
30. STROEMER, R. P.; KENT, T. A.; HULSEBOSCH, C. E. Neocortical neural sprouting, synaptogenesis, and behavioral recovery after neocortical infarction in rats. Stroke, v. 26, n. 11, p. 2135-44, November 1995. ISSN 0039-2499 (Print) 0039-2499.
31. VARKUTI, B. et al. Quantifying the link between anatomical connectivity, gray matter volume and regional cerebral blood flow: an integrative MRI study. PLoS One, v. 6, n. 4, p. e14801, 2011. ISSN 1932-6203.
32. ______. Resting state changes in functional connectivity correlate with movement recovery for BCI and robot-assisted upper-extremity training after stroke. Neurorehabil Neural Repair, v. 27, n. 1, p. 53-62, January 2013. ISSN 1545-9683.
33. WOLF, S. L. On “Effects of forced use on arm function in the subacute phase . . . ” Hammer A M, Lindmark B. Phys Ther. 2009; 89:526-539. In: (Ed.). Phys Ther. United States, v. 89, 2009. p. 993-5; author reply 995-7. ISBN 1538-6724 (Electronic) 0031-9023 (Linking)
34. WOLPAW, J.; WOLPAW, E. W. Brain-Computer Interfaces: Principles and Practice. Oxford University Press, USA, 2012. ISBN 9780195388855. Disponível em: <https://books.google.com/books?id=tC2UzuC_WBQC>.
35. WOLPAW, J. R. Harnessing neuroplasticity for clinical applications. In: (Ed.). Brain. England, v. 135, 2012. p. e215; author reply e216. ISBN 1460-2156 (Electronic) 0006-8950 (Linking).

1. A method of correcting brain impairment to improve body movement, comprising:
analyzing at least one non-impaired brain of a subject during body movement using an rtfMRI device to target brain areas associated with those body movements;
monitoring an impaired brain of a patient at the targeted brain areas using an fNIRS device and generating an output signal from the fNIRS device corresponding to brain activity in the targeted brain areas; and
processing the output signal to produce visual feedback to the patient corresponding to positive or negative feedback relating to an extent of brain activity in the targeted brain areas.

2. The method of claim 1, further including stimulating muscles associated with the body movement using an electrical muscle stimulation device.

3. The method of claim 2, wherein the device is an FES stimulation device.

4. The method of claim 1, wherein the fNIRS device includes a signal generator, a photo multiplier, an amplifier, and an ADC.

5. The method of claim 1, wherein processing the output signal includes compensating for artifacts, including head movement, probe movement, and physiological noise.

6. The method of claim 1, wherein processing the output signal includes converting HbO and HbR values provided by the FNIRS device into brain activity corresponding to brain activity within the targeted brain areas.

7. The method of claim 1, wherein processing the output signal includes producing a graphic, moving visual indicator that is visible to the patient and which corresponds to positive and negative progress towards carrying out a desired brain activity corresponding to the body movement to be improved.

8. The method of claim 6, wherein the visual indicator resembles a thermometer.

9. The method of claim 1, further including processing the output signal to cause a signal from an FES device to stimulate muscles corresponding to brain activity in the targeted brain areas.

10. The method of claim 9, wherein the FES device stimulates muscles to produce muscle movement and afferent nerve impulses which further stimulate the brain, thereby forming a closed-loop feedback system including the brain, the fNIRS, the FES device, and afferent nerves.

11-20. (canceled)

21. A system for correcting brain impairment to improve body movement, comprising:
an rtfMRI device for analyzing at least one non-impaired brain of a subject during body movement to target brain areas associated with those body movements;
an fNIRS device for monitoring an impaired brain of a patient at the targeted brain areas and generating an output signal from the fNIRS device corresponding to brain activity in the targeted brain areas; and
processing means for processing the output signal to produce visual feedback to the patient corresponding to positive or negative feedback relating to an extent of brain activity in the targeted brain areas.

22. The system of claim 21, further including an electrical muscle stimulation device for stimulating muscles associated with the body movement.

23. The system of claim 22, wherein the device is an FES stimulation device.

24. The system of claim 21, wherein the fNIRS device includes a signal generator, a photo multiplier, an amplifier, and an ADC.

25. The system of claim 21, wherein processing the output signal includes compensating for artifacts, including head movement, probe movement, and physiological noise.

26. The system of claim 21, wherein processing the output signal includes converting HbO and HbR values provided by the FNIRS device into brain activity corresponding to brain activity within the targeted brain areas.

27. The system of claim 21, wherein processing the output signal includes producing a graphic, moving visual indicator that is visible to the patient and which corresponds to positive and negative progress towards carrying out a desired brain activity corresponding to the body movement to be improved.

28. The system of claim 26, wherein the visual indicator resembles a thermometer.

29. The system of claim 21, wherein the output signal is processed to cause a signal from an FES device to stimulate muscles corresponding to brain activity in the targeted brain areas.

30. The method of claim 29, wherein the FES device stimulates muscles to produce muscle movement and afferent nerve impulses which further stimulate the brain, thereby forming a closed-loop feedback system including the brain, the fNIRS, the FES device, and afferent nerves.

31-32. (canceled)
